(19) United States
(12) Patent Application Publication
(10) Pub. No.: US 2004/0015229 A1
(43) Pub. Date: Jan. 22, 2004

(54) Vascular stent with radiopaque markers

(75) Inventors: John D. Fulkerson, Plantation, FL (US); Thomas O. Bales, Coral Gables, FL (US); Scott L. Jahrmarkt, Miami Beach, FL (US)

Correspondence Address: Gordon & Jacobson, P.C., 65 Woods End Road, Stamford, CT, 06905, US

(73) Assignee: SYNTHEON, LLC

(21) Appl. No.: 10/201,504

(22) Filed: Jul. 22, 2002

(51) Int. Cl. A61F 2/06

(52) U.S. Cl. 623/1.22; 623/1.34; 600/431

(57) ABSTRACT

A stent includes a marker mount defined by a pair of fingers. The fingers are preferably oriented parallel to the longitudinal axis of the stent. The fingers preferably each include a retainer at their respective ends, and the retainers of each pair of fingers preferably define a composite barb. A tubular marker can be forced over the bifurcated barb and onto the fingers. The marker is preferably provided with a ceramic coating to prevent galvanic corrosion between the stent and the marker.

### BACKGROUND OF THE INVENTION

1. Field of the Invention

This invention relates broadly to arterial prostheses. More particularly, this invention relates to vascular stents.

2. State of the Art

Transluminal prostheses are widely used in the medical arts for implantation in blood vessels, biliary ducts, or other similar organs of the living body. These prostheses are commonly known as stents and are used to maintain, open, or dilate tubular structures.

Stents are either balloon expandable or self-expanding. Balloon expandable stents are typically made from a solid tube of stainless steel. Thereafter, a series of cuts are made in the wall of the stent. The stent has a first smaller diameter configuration which permits the stent to be delivered through the human vasculature by being crimped onto a balloon catheter. The stent also has a second, expanded diameter configuration, upon the application, by the balloon catheter, from the interior of the tubular shaped member of a radially, outwardly directed force.

Self-expanding stents act like springs and recover to their expanded or implanted configuration after being compressed. As such, the stent is inserted into a blood vessel in a compressed state and then released at a site to deploy into an expanded state. One type of self-expanding stent is composed of a plurality of individually rigid but flexible and elastic thread elements defining a radially self-expanding helix. This type of stent is known in the art as a “braided stent”. Placement of such stents in a body vessel can be achieved by a device which comprises an outer catheter for holding the stent at its distal end, and an inner piston which pushes the stent forward once it is in position. However, braided stents have the disadvantage that they typically do not have the necessary radial strength to effectively hold open a diseased vessel. In addition, the plurality of wires or fibers used to make such stents could become dangerous if separated from the body of the stent, where it could pierce through the vessel.

Therefore, recently, self-expanding stents cut from a tube of superelastic metal, e.g., a nickel-titanium alloy, have been manufactured. These stents are crush recoverable and have relatively high radial strength.

Typically, stent materials such as stainless steel and nickel titanium alloys are not readily perceptible when medical imaging devices, such as fluoroscopes, are used to view the site where the stent has been implanted. To enhance the radiopacity of surgical stents, it is known in the prior art to provide a radiopaque marker on the stent which is clearly identifiable when a fluoroscope or other imaging device is used. Such radiopaque-marker stents taught in the prior art have suffered from a number of drawbacks.

One type of radiopaque marker is welded, brazed or diffusion bonded to couple the marker with the stent. However, this is a permanent, irreversible process. As such, if there are multiple markers to be attached, and if one is improperly attached, the entire stent is made unusable. In addition, there is no alternative but to have the marker material electrically attached to the stent, with the possible result of galvanic corrosion. In any metallurgical joining of marker material to a stent, there is an unavoidable intermetallic alloying which occurs in the interface zone. In most cases this zone has reduced properties such as elasticity (or superelasticity), brittleness, and corrosion resistance. Furthermore, such weld or braze joints are very difficult to inspect and can often contain latent defects which would allow separation of the marker from the stent, resulting in embolization.

A second type of marker, e.g., as seen in U.S. Pat. No. 6,022,374, is a malleable radiopaque marker that can be inserted into a recess in a stent and deformed in such a way that it is anchored in place. A disadvantage of this technique is that the overall structure associated with the marker is much bigger than the marker, since stent material (which is not radiopaque) must substantially surround the radiopaque material of the marker. Again, galvanic corrosion is a possible problem.

A third type of marker can be constructed of a radiopaque material and then snapped into a receiver formed of the stent material. This design is particularly adaptable to stents constructed of superelastic material, because the necessary manufacturing tolerances of markers and stents are a relatively large fraction of the size of such markers, and it is necessary to design to a large amount of stretch in the stent material to accommodate the variation of part sizes. However, the structure is large because the radiopaque material must be surrounded by stent material which is not radiopaque.

A fourth type of marker is a tubular marker that can be crimped around a portion of the stent. This design is common in stents formed of wire, since it is easier to load a tube of radiopaque material onto a wire while it is being wound into the stent form. In such a configuration, the marker is relatively narrow, typically fifty microns thick surrounding a wire with a width around one hundred microns. Since wire stents are usually made of wire which is of round cross-section, it is not practical to make a marker which is substantially wider than the diameter of the wire. A marker which is wide and flat would be at risk of rotating around the axis of a round wire, and it is desirable for a flat marker to remain in the cylindrical plane of the surface of the stent. Also, the marker would adversely affect the flexibility of a stent strut if it were located in an area where the strut flexes. In addition, crimping a marker about an internal strut fails to identify the ends of stent under radioimaging.

Moreover, if the stent is constructed from a laser-cut tube, it would be impossible to place a tubular marker around one of the flexible struts, since these struts have no “free” ends over which the marker could be slipped. Of course, a split tubular marker could be used, but such a marker would be difficult to manufacture and attach, and it would adversely affect the flexibility of the strut.

Furthermore, it is difficult to create a crimped-on marker which is electrically (and galvanically) isolated from the stent, because a malleable insulating layer would have to be interposed between the marker and the stent, and such a layer would have to be a separate, probably polymeric material.

As a fifth type of marker, a radiopaque substance can be deposited on a stent by electroplating, chemical vapor deposition, or other such coating processes to create an overall (or selective) coating of the radiopaque substance on the surface of the stent. There are advantages to such a method in that it allows overall visualization of the stent, but problems remain with galvanic corrosion. Even worse, there are no radiopaque elements or alloys which have elastic elongation compatible with superelastic stents. If a less-elastic material is coated onto a superelastic stent and the stent is then highly flexed (as occurs during implantation and possibly when exposed to in-vivo pressure pulsations), the coating material is likely to crack, spall, delaminate, or otherwise fail. It is possible to restrict the applied radiopaque material to non-flexing areas of the stent, but all the advantages of metallurgically-bonded dissimilar materials (corrosion, separation, etc.) remain.

### SUMMARY OF THE INVENTION

It is therefore an object of the invention to provide a stent having a radiopaque marker wherein the interaction of the marker and the stent material will not cause galvanic corrosion.

It is another object of the invention to provide a stent having radiopaque markers which indicate the extremities of the stent.

It is a further object of the invention to provide a stent having a radiopaque marker which does not adversely affect radial expansion of the stent.

It is an additional object of the invention to provide a stent having a radiopaque marker which is relatively easy to attach to the stent.

It is also an object of the invention to provide a stent having radiopaque markers which are securely held by the stent, but can be relatively easily detached from the stent.

In accord with these objects, which will be discussed in detail below, a stent is provided with markers that are positively retained on the stent, that can be replaced during manufacturing if desired, and that are electrically insulated from the stent by a ceramic coating on the marker.

According to a preferred aspect of the invention, the markers are tubular in configuration, and the ceramic coating is an oxide.

According to another preferred aspect of the invention, the stent is comprised of an elastic or superelastic material and includes, at each of its ends, at least one marker mount defined by a pair of fingers. The fingers are preferably oriented parallel to the longitudinal axis of the stent. The fingers preferably each include a retainer at their respective ends, and the retainers of each pair of fingers preferably together define a composite barb. A tubular marker can be forced over the composite barb, moving the fingers of a pair closer together, and further moved onto the remaining portion of the finger, permitting the fingers to spring back apart such that the composite barb operates to retain the marker. The marker can be removed by pressing the fingers toward each other to minimize the effective size of the composite barb and thereby release the marker.

According to another aspect of the invention, the marker mount and marker assembly can also be provided to inelastic stent designs. In such stents, the markers are placed over the fingers, and the fingers are then plastically deformed to retain the markers.

Additional objects and advantages of the invention will become apparent to those skilled in the art upon reference to the detailed description taken in conjunction with the provided figures.

### BRIEF DESCRIPTION OF THE DRAWINGS

FIG. 1 is a broken perspective view of an end of a stent in an unexpanded state and having four marker mounts at each of its ends according to the invention;

FIG. 2 is broken flattened view of a stent in an unexpanded state that has been cut parallel to its longitudinal axis and laid flat, the stent having six marker mounts at each of its ends according to the invention;

FIG. 3 is a side section view of a marker according to the invention;

FIG. 4 is an end view of a marker according to the invention;

FIG. 5 is a view similar to FIG. 1 shown with markers mounted on the marker mounts according to the invention; and

FIG. 6 is broken flattened view of a stent in an unexpanded state having a marker mounts according to a second embodiment of the invention and shown with a marker on one of the mounts.

### DETAILED DESCRIPTION OF THE PREFERRED EMBODIMENTS

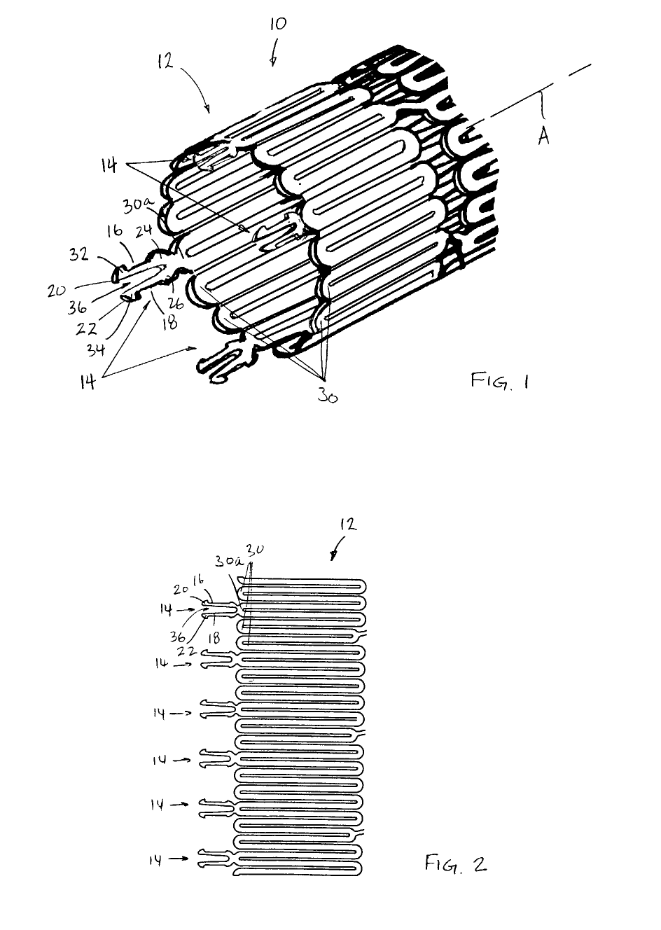

Turning now to FIGS. 1 and 2, a stent according to one embodiment of the invention is shown. The stent 10 is preferably a cut tubular stent that is either balloon expandable or self-expanding. In accord with the invention, each end 12 of the stent 10 is provided with preferably a plurality of marker mounts 14, e.g., four mounts (FIG. 1) or six mounts (FIG. 2).

Each marker mount 14 is defined by a pair of fingers 16, 18 preferably oriented substantially parallel to the longitudinal axis A of the stent. Each finger 16, 18 includes a free end 20, 22 defining a slot or space 36, and an opposite end 24, 26 that is preferably joined to one loop 30a of many cylindrical or helical loops 30 forming the end 12 of the stent. The free end 20, 22 of at least one of the fingers and preferably each finger 16, 18 defines an enlarged retainer portion or barb 32, 34. Each retainer portion 32, 34 preferably extends at an angle relative to an axis of its finger. As the free ends 20, 22 of the fingers 16, 18 can be forced toward each other (i.e., to close the slot 36), the two retainer portions of a marker mount preferably together define a compressible composite or bifurcated barb. The fingers are each preferably rectangular in cross-section. According to a preferred, but exemplar configuration, each finger is approximately 75 microns wide and 200 microns thick.

Shown best in FIG. 2, according to a first embodiment of the invention, the stent loops 30 at the end 12 all terminate at about the same axial location, and the marker mounts 14 extend from that location. In addition, as previously mentioned, the fingers 16, 18 are separated by a generally V-shaped space 36 that permits relatively large movement of the free ends 20, 22 of the fingers toward each other.

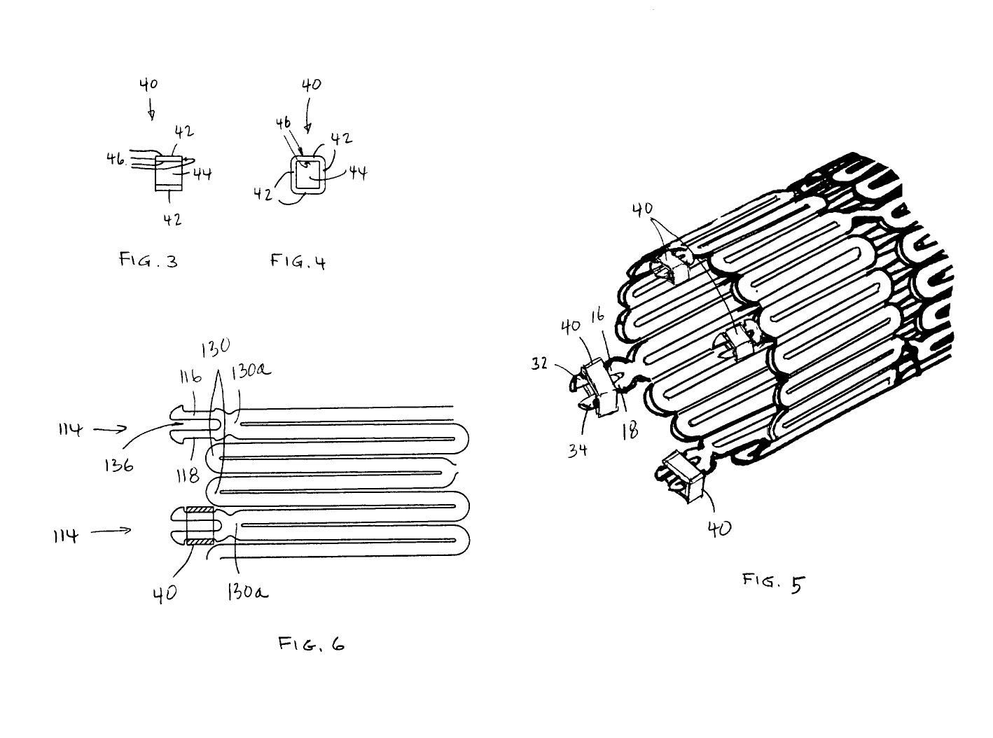

Referring to FIGS. 3 and 4, a marker 40 according to the invention is provided for use with the stent 10. The marker 40 is a tube having rectangular cross section. The relatively flat sides 42 which define a rectangular internal opening 44 of the marker are sized to closely receive the fingers 16, 18 of the marker mount 14. In addition, the sides are preferably uninterrupted; i.e., the marker is not a slit tube. The marker 40 is preferably made of either tantalum, zirconium, hafnium, gold or platinum and, according to one exemplar embodiment, preferably has a thickness of substantially 50 microns (i.e., 50±20%), a length of substantially 300 microns (300±20%), and defines an inside dimension of substantially 220 microns square (220±20%). The marker 40 is preferably thermally processed by heating in air to create an oxide film 46 over its surfaces.

When the marker mount 14 of the invention is provided on a self-expanding stent, such as made of a nickel-titanium superelastic alloy, the marker 40 can be forced over the composite barb defined by the retainer portions 32, 34, as such force will move the fingers 16, 18 of a mount 14 closer together so that the retainer portions fit through the internal opening 44 of the marker. Referring to FIG. 5, the marker 40 is then moved further onto the fingers 16, 18 beyond the retainer portions 32, 34, permitting the fingers to spring back apart such that the composite barb defined by the retainer portions operates to retain the marker. The interfering cross-sectional shapes of the fingers 16, 18 and the internal opening 44 prevent rotation of the marker 40 on the mount 14. The oxide film 46 on the surfaces of the marker 40 is a ceramic which operates to insulate the marker from the stent material, and thereby decreases the likelihood of galvanic corrosion from contact between the marker and stent. It also provides a hard surface which can be forced over the bifurcated barb without damage to itself.

If necessary, each marker 40 can be individually removed from its mount 14 by pressing the fingers 16, 18 of the mount toward each other to minimize the effective size of the composite barb (i.e., to make it smaller than the internal opening 44 of the marker) and thereby release the marker.

Turning now to FIG. 6, an alternate embodiment of the marker mount 114 is shown. The marker mount 114 include fingers 116, 118 defining a U-shaped space 136 therebetween, rather than the V-shape space 36 shown in FIG. 2. In addition, the loops 130a of the stent provided with the marker mounts 114 are relatively shorter than the other loops 130. As such, the markers 40 seat closer to adjacent loops 130 of the stent.

While the marker mount and marker assembly is primarily intended for use on a superelastic stent (which typically will allow up to eight percent strain during manufacture), the marker mounts may also be constructed on stents of normal elastic materials, such as MP-35N or platinum-iridium, or using plastically-deformable materials, such as stainless steel. In the case of a plastically-deformable material, the tubular marker is placed over fingers of a generally similar design (the enlarged retainer portions not being necessary), and the free ends of the fingers are then plastically deformed outwardly to retain the marker.

There have been described and illustrated herein several embodiments of a stent provided with markers. While particular embodiments of the invention have been described, it is not intended that the invention be limited thereto, as it is intended that the invention be as broad in scope as the art will allow and that the specification be read likewise. Thus, while particular marker mounts have been disclosed, it will be appreciated that other marker mount configurations can be used as well. For example, while the retainer portions of the fingers have been described as defining a bifurcated barb at the end of each pair of fingers, it is recognized that only one of the fingers need have a barb structure or be plastically deformed after marker placement to retain the marker. Also, while particular stent materials, marker materials, and dielectric ceramic coatings have been disclosed, it will be recognized that other suitable materials, dielectric coatings, etc. can be used. Furthermore, while the fingers preferably extend parallel to the stent axis, it is appreciated that the fingers may extend at an angle, e.g., between 0° and 90°, relative to the stent axis. Moreover, while the fingers as shown are preferably parallel to each other, they may be slightly angled relatively to each other (e.g., between 0° and 10°) and still be considered substantially parallel for purposes of the invention. It will therefore be appreciated by those skilled in the art that yet other modifications could be made to the provided invention without deviating from its spirit and scope as claimed.

What is claimed is:

1. A stent for insertion into a vessel of a patient, said stent adapted to receive a marker element, comprising:
a tubular member having a first smaller diameter configuration for insertion into the vessel, and a second larger diameter configuration for deployment within the vessel,
said tubular member including two ends, at least one of said ends including at least one marker mount extending therefrom,
each said marker mount including a pair of fingers, at least one of the fingers having an enlarged retainer portion, and at least one of said fingers of said pair of fingers adapted to be moved relative to the other of said fingers of said pair of fingers to receive a marker over said pair of fingers.

2. A stent according to claim 1, wherein:
said fingers are one of elastically deformable and plastically deformable.

3. A stent according to claim 1, wherein:
said tubular member comprises one of a superelastic alloy, an elastic alloy, and a plastically deformable material.

4. A stent according to claim 1, wherein:
said tubular member includes a plurality of cylindrical or helical loops, and each said marker mount is coupled to one of said loops.

5. A stent according to claim 1, wherein:
said fingers are substantially parallel to each other.

6. A stent according to claim 1, wherein:
said tubular member defines a longitudinal axis and said fingers are substantially parallel to said longitudinal axis.

7. A stent according to claim 1, wherein:
said fingers are spaced apart with a U-shaped space.

8. A stent according to claim 1, wherein:
said fingers are spaced apart with a V-shaped space.

9. A stent according to claim 1, wherein:
each of said fingers includes a retainer portion.

10. A stent according to claim 9, wherein:
said retainer portions of each said pair of fingers together define a bifurcated barb.

11. A stent according to claim 1, wherein:
said tubular member includes a plurality of marker mounts about said one of said two ends of said tubular member

12. A stent according to claim 1, wherein:
said tubular member includes two ends, each of said ends provided with at least one marker mount.

13. A stent according to claim 1, further comprising:
a radiopaque marker element coupled about said pair of fingers of said marker mount.

14. A stent according to claim 13, wherein:
said marker element is tubular.

15. A stent according to claim 14, wherein:
said marker element has a rectangular external cross-sectional shape.

16. A stent according to claim 14, wherein:
said marker element has a rectangular internal cross-sectional shape.

17. A stent according to claim 14, wherein:
said marker has flat sides.

18. A stent according to claim 13, wherein:
said marker element is made of one of tantalum, zirconium, hafnium, and platinum.

19. A stent according to claim 13, wherein:
said marker element has a dielectric coating thereon.

20. A stent according to claim 19, wherein:
said marker element is made of a metal, and said dielectric coating is an oxide of said metal.

21. A stent according to claim 1, wherein:
movement of one of said fingers toward the other of said fingers permits removal of said marker element from about said pair of fingers.

22. A stent for insertion into a vessel of a patient, said stent adapted to receive a marker element and having a longitudinal axis, comprising:
a tubular member having a first smaller diameter configuration for insertion into the vessel, and a second larger diameter configuration for deployment within the vessel,
said tubular member including two ends, at least one of said ends including at least one marker mount extending therefrom,
each said marker mount including a pair of fingers extending substantially parallel to each other and to said longitudinal axis of said stent, each of said fingers having an enlarged retainer portion and said retainer portions together defining a bifurcated barb, and at least one of said fingers of said pair of fingers adapted to be moved relative to the other of said fingers of said pair of fingers to receive a marker over said pair of fingers

23. A stent according to claim 22, further comprising:
at least one radiopaque marker, each of said at least one marker being received on one of said pair of fingers of one of said marker mounts.

24. A stent for insertion into a vessel of a patient, comprising:
a) a metal or metal alloy tubular member having a first smaller diameter configuration for insertion into the vessel, and a second larger diameter configuration for deployment within the vessel, said tubular member including two ends, at least one of said ends including a marker mount extending therefrom; and
b) a metal or metal alloy radiopaque marker mounted to said marker mount, said marker being electrically shielded from said marker mount by a dielectric coating on said marker, said dielectric coating being an oxide of said metal or metal alloy of said radiopaque marker.

25. A stent according to claim 24, wherein:
said tubular member includes a plurality of marker mounts about said one of said two ends of said tubular member, each provided with a radiopaque marker.

26. A stent according to claim 24, wherein:
said tubular member includes two ends, each of said ends provided with at least one marker mount, and each of said marker mounts provided coupled to a radiopaque marker.

27. A stent according to claim 24, wherein:
said metal or metal alloy of said tubular member includes one of tantalum, zirconium, and hafnium.

28. A stent according to claim 24, wherein:
said marker is received over said marker mount.

29. A stent according to claim 30, wherein:
said marker has a tubular construct.

30. A radiopaque marker for an implantable medical device, comprising:
a tubular element comprising a radiopaque material and having a substantially rectangular internal and external cross-sectional shapes and substantially flat sides,
said marker having a dielectric coating thereon.

31. A radiopaque marker according to claim 30, wherein:
said sides of said tubular element are uninterrupted.

32. A radiopaque marker according to claim 30, wherein:
said tubular element is made of one of tantalum, zirconium, hafnium, gold and platinum.

33. A radiopaque marker according to claim 30, wherein:
said dielectric coating is an oxide of said radiopaque material.

34. A radiopaque marker according to claim 30, wherein:
said marker has a thickness of substantially 50 microns, a length of substantially 300 microns, and an inside dimension of substantially 220 microns square.
